(19) United States
(12) Patent Application Publication
(10) Pub. No.: US 2008/0015693 A1
(43) Pub. Date: Jan. 17, 2008

(54) Spinal Arthroplasty System

(76) Inventor: Regis Le Couedic, Andresy (FR)

Correspondence Address: LEFFERT JAY & POLGLAZE, P.A., P.O. BOX 581009, MINNEAPOLIS, MN, 55458-1009, US

(21) Appl. No.: 11/597,758

(22) PCT Filed: May 26, 2005
(86) PCT No.: PCT/FR05/01301

§ 371 Date: Nov. 27, 2006

(30) Foreign Application Priority Data
May 27, 2004 (FR) 0405720

(51) Int. Cl. A61F 2/44

(52) U.S. Cl. 623/17.11; 623/17.16

(57) ABSTRACT

The invention relates to a spinal arthroplasty system for putting into place between two contiguous vertebrae (V1, V2). The arthroplasty system comprises:
an at least partial prosthesis (12) for the intervertebral disk; and
an intervertebral implant (14) comprising:
   a spacer (20) for placing between the spinous processes of the two vertebrae, said spacer including at each of its ends a housing (22, 24) for receiving one of the spinous processes; and
   at least one tie (26, 28) for holding each spinous process in a housing of said spacer.

The present invention provides a spinal arthroplasty system.

The principle of replacing an intervertebral disk by a mechanical type prosthesis in order to perform spinal arthroplasty is known and described in numerous documents.

This operation seeks to treat pathologies of degeneration of the intervertebral disk and it can be implemented via an anterior surgical approach or a posterior approach.

Another technique for implementing treatment of that type of pathology consists in using implants that make it possible to replace only the nucleus pulposus of the intervertebral disk. Such implants are said to be nucleus prostheses. The prosthesis is inserted in the central portion of the intervertebral disk after removing its own nucleus. Such prostheses are generally made of a hybrophilic material that expands up to a final size over the 24 or 48 hours following the operation, and that presents viscoelastic properties that tend to reproduce the mechanical behavior of the nucleus.

Nevertheless, the indications relating to the use of intervertebral disk prostheses, whatever the approach used, or using a nucleus prosthesis, are constrained by the state of the posterior structures at the level of the spine that is to be treated. The term “posterior structure” is used to designate the posterior joints formed between the joint facets of the vertebrae disposed above and below the patient's disk. When the posterior joints are themselves suffering from degeneration, the indication comprising an intervertebral disk prosthesis or a nucleus prosthesis is often deemed to be too severe to enable a partial or total disk prosthesis to be used under good conditions and with a real chance of success. This is due in particular to the fact that both when using a mechanical intervertebral prosthesis and when using a nucleus prosthesis, those devices seeking to replace at least part of the natural intervertebral disk do not include any mechanical stops on the relative movements of the adjacent vertebrae. Under such circumstances, the vertebrae themselves, given their degeneration, do not constitute such mechanical stops for limiting relative movement. The surgeon thus often has no alternative other than to proceed with fusion at the level in question of the spine, e.g. using a system that includes pedicular screws connected to rods for preventing the two vertebrae from moving, which can naturally diminish patient comfort.

An object of the present invention is to provide a spinal arthroplasty system that makes it possible to use an intervertebral disk prosthesis or a nucleus prosthesis even in association with severe degeneration of the posterior joint elements of the vertebrae located above and below the diseased disk.

To achieve this object of the invention, the spinal arthroplasty system for putting into place between two contiguous vertebrae is characterized in that it comprises an at least partial intervertebral disk prosthesis selected from the group comprising ball-and-socket type mechanical prostheses and intervertebral disk nucleus prostheses, together with a spacer for placing between the spinous processes of the two vertebrae, said spacer including at each of its ends a housing for receiving one of the spinous processes, and at least one tie for holding each process in a housing of said spacer.

It will be understood that by using the intervertebral implant placed between the spinous processes of the vertebrae disposed on either side of the diseased disk, a mechanical stop function is implemented on relative movement that the vertebrae suffering from degeneration are themselves no longer capable of implementing. It is thus possible to make use under good conditions of a nucleus implant or of a ball-and-socket type mechanical implant for the intervertebral disk. The intervertebral implant spacer serves to limit relative movements between the two vertebrae in the various planes of mobility.

In a first embodiment, the system includes an intervertebral disk prosthesis comprising a first assembly presenting a fastener face for fastening to a vertebra and an active face in which a portion is in the form of a concave first substantially spherical cap, and a second assembly presenting a fastener face for fastening to the other vertebra and an active face having a portion in the form of a convex second substantially spherical cap for co-operating with the first spherical cap. As is likewise well known, the disk prosthesis constituted by the two elements having complementary spherical caps allows for relative movements to be performed between the two vertebrae that are close to natural relative movements.

In a second embodiment, the system includes a nucleus prosthesis that is put into place in the natural intervertebral disk after its own central portion has been removed.

Other characteristics and advantages of the invention appear better on reading the following description of various embodiments of the invention given as non-limiting examples. The description refers to the accompanying drawings, in which:

FIG. 1 is a simplified view of a first embodiment of the invention with an intervertebral disk prosthesis;

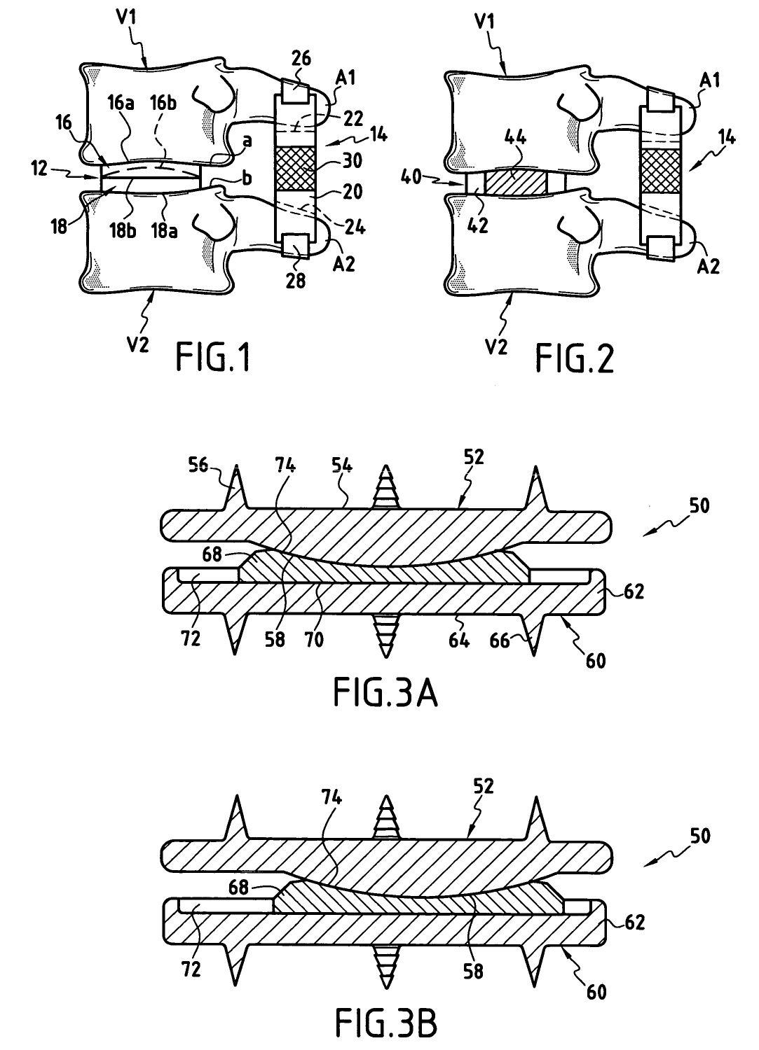

FIG. 2 is a simplified view of a second embodiment of the invention using a nucleus prosthesis;

FIGS. 3A and 3B are diametrical sections showing a first embodiment of the intervertebral disk prosthesis;

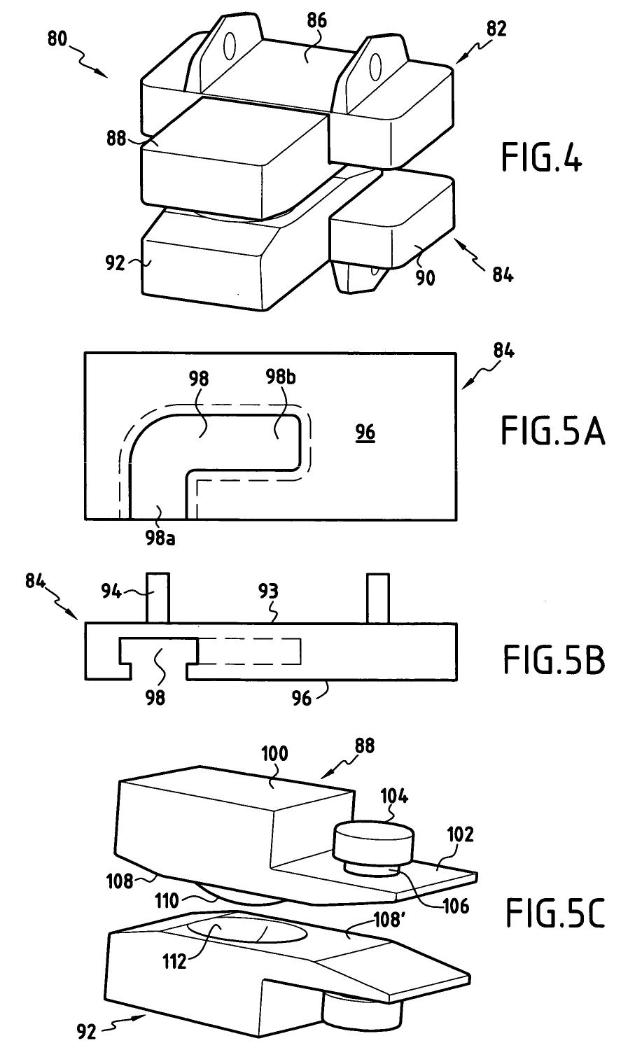

FIG. 4 is a perspective view of a second embodiment of the intervertebral disk prosthesis;

FIGS. 5A, 5B, and 5C show different portions of the intervertebral disk prosthesis shown in FIG. 4;

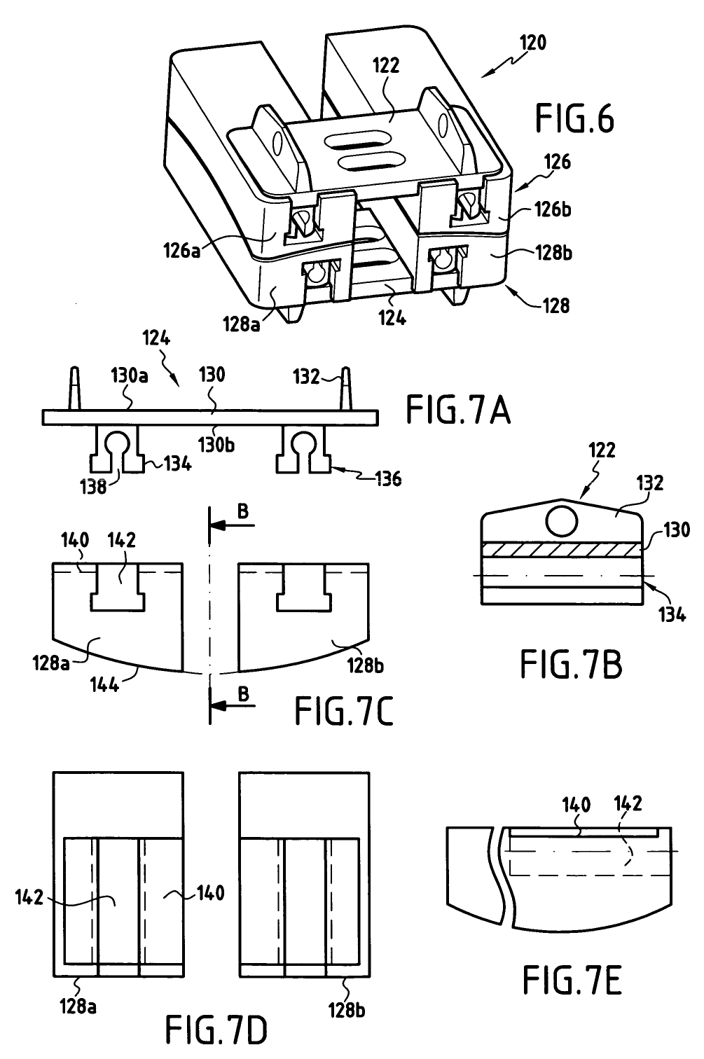

FIG. 6 is a perspective view of a third embodiment of the intervertebral disk prosthesis;

FIGS. 7A to 7E show details of various portions of the intervertebral disk prosthesis shown in FIG. 6;

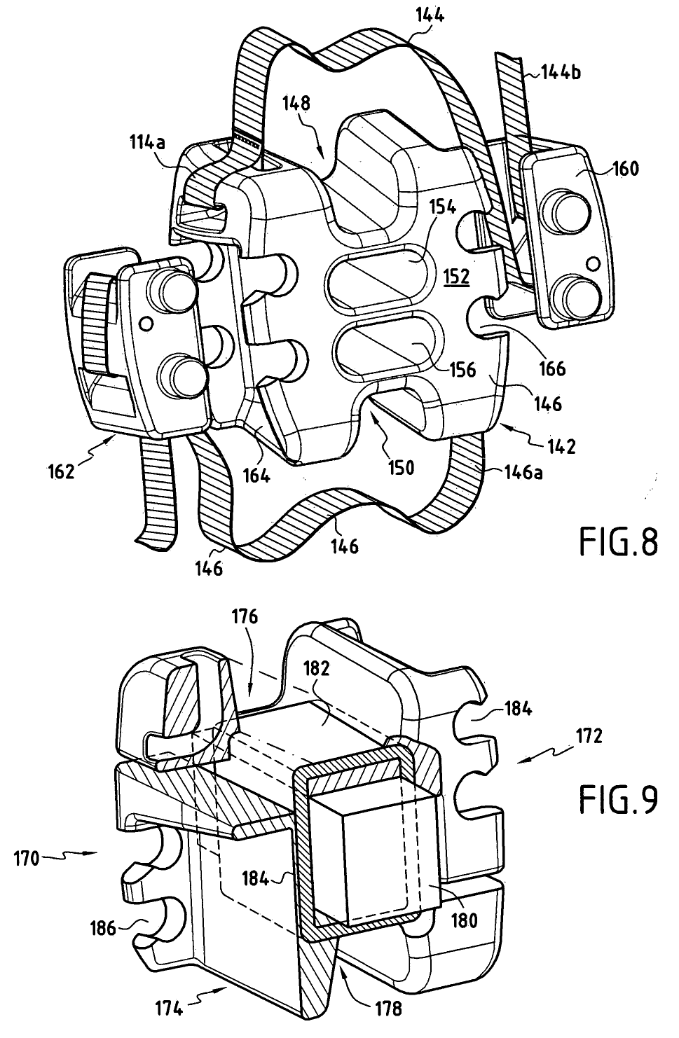

FIG. 8 is a perspective view of a first embodiment of the intervertebral implant; and

FIG. 9 is a perspective view of a second embodiment of the intervertebral implant.

FIG. 1 is a simplified view of a first embodiment of a spinal arthroplasty system of the invention.

In the figure, there can be seen two vertebrae V1 and V2 and their spinous processes A1 and A2. In this embodiment, the spinal arthroplasty system is constituted by an intervertebral disk prosthesis 12 and by an intervertebral implant 14. The intervertebral disk prosthesis 2 is mounted between the plates a and b of the vertebrae V1 and V2. The prosthesis 12 is essentially constituted by two elements 16 and 18, each presenting one face 16a, 18a for fastening to a plate of a vertebra, and another face 16b, 18b, which other faces define common contact surfaces that are in the form of spherical caps. By means of the contact surfaces 16b and 18b, the two prosthesis elements can move relative to each other, thus enabling the vertebrae V1 and V2 to perform at least some of their natural relative movements.

The intervertebral implant 14 is essentially constituted by a spacer 20 that presents grooves 22 and 24 at its ends for receiving the spinous processes Al and A2. The spacer 14 is secured to the processes A1 and A2 by means of two straps 26 and 28 whose ends are secured to the spacer 20 and that surround the processes. The central portion 30 of the spacer 14 is preferably elastically deformable, thus allowing a certain amount of relative movement between the vertebrae V1 and V2.

It will be understood that in the circumstances taken into consideration for the invention, i.e. when the posterior joints of the vertebrae are suffering degeneration, the intervertebral implant 14 serves to limit relative movements between the two vertebrae in a way that would normally have been performed by the posterior joints while they were in good condition. It will also be understood that combining the intervertebral disk prosthesis and the intervertebral implant makes it possible to solve the problem of replacing the natural intervertebral disk under the particular conditions under consideration.

FIG. 2 is likewise a simplified diagram showing a second embodiment of the spinal arthroplasty system of the invention. The natural intervertebral disk 40 presents more limited degeneration. In this circumstance, instead of using the prosthesis 12, the peripheral portion 42 of the natural disk is conserved and its nucleus pulposus is replaced, using a nucleus prosthesis 44 which takes the place of the central portion of the natural disk. Nucleus prostheses are themselves known as mentioned above and they are generally made of a hydrophilic material that expands up to its final size during the 24 or 48 hours following the operation. These nucleus prostheses that constitute in fact a partial prosthesis for the natural disk present viscoelastic characteristics that tend to reproduce the mechanical behavior of the nucleus. It should be emphasized that it is possible to use a nucleus prosthesis only if the posterior structures of the vertebrae are in perfect condition since the nucleus prosthesis does not provide any guidance for relative movements between the two vertebrae, whereas the intervertebral disk prosthesis enables a limited amount of guidance to be provided.

Nucleus prostheses for intervertebral disks are described in European Spine Journal, Volume 11, Supplement 2, October 2002 and in SPINE, Volume 27 (11), Jun. 1, 2002, pp. 1245 to 1247.

In this second embodiment of the spinal arthroplasty system, the nucleus prosthesis 44 is associated with an intervertebral implant 14 identical to that described with reference to FIG. 1. As explained above, the intervertebral implant is placed between the spinous processes A1 and A2 serving to limit the possibilities for relative movement between the two vertebrae so that, with respect to this function, the implant takes the place of the posterior structures of the vertebrae that have degenerated.

It will be understood that in the first embodiment of the invention the spinal arthroplasty system is constituted by an intervertebral disk prosthesis that can present a large number of different structures and by an intervertebral implant for limiting relative movements, that can likewise present numerous different structures.

In the description below, various intervertebral disk prostheses suitable for use in the invention are described, as are various intervertebral implants likewise suitable for use with the invention.

FIGS. 3A and 3B show a first embodiment of an intervertebral disk implant, given reference 50. The first assembly 52 of the prosthesis 50 has a fastener face 54 provided with anchor means 56 for anchoring in the plate of the vertebra, and a contact face 58 in the form of a spherical cap. The second element 60 of the prosthesis is constituted by a fastener part 62 having a fastener face 64 provided with anchor members such as 70, and by a contact part 68. The part 58 has a sliding face 70 suitable for co-operating with the bottom of a spot face 72 formed in the first part 62. The second part 68 also has an active face 74 in the form of a spherical cap for co-operating with the surface 58 in the form of a spherical cap belonging to the first assembly 52.

It will be understood that this intervertebral disk prosthesis accommodates not only rolling relative movements via the presence of two spherical caps, but also movement in translation. Such an intervertebral disk prosthesis is described in greater detail in PCT application WO 00/53127. Other intervertebral implant prostheses are described by way of example in European patent application EP 0 176 728.

Under all circumstances, the intervertebral prosthesis of the type described above is put into place via an anterior approach. It will be understood that as a general rule such a solution is sub-optimal since the intervertebral implant 14 needs to be put into place via a posterior approach.

To remedy that drawback, it is desirable to use an intervertebral disk prosthesis that is suitable for being put into place via a posterior approach. Two preferred examples of a prosthesis of this type are described below with reference to FIGS. 4 and 5 and with reference to FIGS. 6 and 7.

With reference initially to FIGS. 4 and 5A to 5C, an intervertebral disk prosthesis 80 is described that can be put into place via a posterior approach. The prosthesis 80 comprises a first prosthesis assembly 82 and a second prosthesis assembly 84. The first assembly 82 is constituted by a first fastener element 86 and by a first prosthesis element 88, while the second assembly 84 is constituted by a second fastener element 90 and by a second prosthesis element 92.

The fastener element 84 is described with reference to FIGS. 5A and 5B, and the fastener element 86 is identical thereto. The element 84 is generally in the form of a rectangular plate having a fastener face 92 provided with anchor means 94 for anchoring in the plate of the vertebra, and an opposite face 96 for co-operation purposes.

In the co-operation face 96 there is formed a slot 98 that opens out into the face 96 and that presents a right section that is T-shaped, as can be seen in FIG. 5B. The slot 98 presents an engagement portion 98a that opens out into one of the sides of the part 84, and a second portion 98b that is perpendicular thereto.

FIG. 5C shows the prosthesis elements 88 and 92. The prosthesis element 88 has a co-operation face 100 with a set-back portion 102 having a locking member 104 formed therein that is constituted by a stud with a smaller-diameter portion 106 connecting it to the surface 102. The dimensions of the stud 104 are determined in such a manner as to enable it to be engaged in the slot 98. The prosthesis element 88 also has an active face 108 that essentially defines a surface in the form of a convex spherical cap 110.

The prosthesis element 92 presents the same architecture as the prosthesis element 88, with the exception of its active face 108′ defining a surface in the form of a concave spherical cap 112 suitable for co-operating with the convex spherical cap 110 of the prosthesis element 88.

The intervertebral disk prosthesis 80 is put into place via a posterior approach as follows:

Initially, the surgeon puts into place the fastener elements 82 and 84 either successively or simultaneously. This can be done from a posterior approach because of the relatively small size of the fastener elements. Thereafter, the surgeon introduces successively each prosthesis element 88 and 92 in such a manner that the anchor stud 104 penetrates into the insertion portion of the slot 98 of the corresponding fastener element. Then by pivoting through 90°, the surgeon moves the stud 104 into the second portion 98b of the slot 98 until it becomes held therein. This enables each prosthesis element to be locked onto a fastener element. It should be emphasizes that by the configurations of the respective locking elements (slot 98, stud 104), the locking of the prosthesis elements on the fastener elements is performed solely by moving the prosthesis elements in a plane that is perpendicular to the common axis of the two vertebrae between which the prosthesis is to be put into place.

With reference to FIGS. 6 and 7A to 7E, a second example of an intervertebral disk prosthesis is described that can be put into place via a posterior approach.

FIG. 6 shows that the prosthesis 120 is constituted by two fastener elements 122 and 124 and by two prosthesis elements 126 and 128, each prosthesis element being constituted by two distinct parts 126a & 126b for the element 126, and 128a & 128b for the element 128.

FIGS. 7A and 7B show the fastener element 122, the fastener element 124 being identical thereto. The fastener element 122 is constituted by a substantially rectangular plate 130 whose fastener face 130a is fitted with anchor members such as 132. The other face 130b of the plate 130 is fitted with two locking members 134 and 136. Each locking member 134, 136 is in the form of a bar of T-shaped right section. Each locking member preferably includes a longitudinal slot 138 for conferring a certain amount of elasticity to the locking members.

FIGS. 7C to 7E show the parts 128a and 128b of the prosthesis element 128.

Each part 128a and 128b of the prosthesis element 128 includes in its co-operation face 140 an open T-shaped slot 142 suitable for receiving the locking members 134 and 136. It will be understood that when the parts 128a and 128b are secured to the plate 130, these parts are prevented from moving relative to each other. The contact faces 144 of the parts 128a and 128b define a portion of a spherical cap. The two spherical cap portions defined in this way belong to a common spherical cap when the parts 128a and 128b are secured to each other with the help of the fastener element part 124. Similarly, the parts 126a and 126b present active faces which together define a concave spherical cap.

It would be understood that the above-described embodiment of the intervertebral disk prosthesis is particularly well adapted to being put into place via a posterior approach. The surgeon can initially put the two fastener elements 122 and 124 into place on the vertebral plates, either simultaneously or in succession. Thereafter, the parts 126a and 126b constituting the prosthesis element 126 are put into place on either side of the spinal cord and are locked to the fastener element 122, and then in the same manner, the parts 128a and 128b of the prosthesis element 128 are put into place and locked to the fastener element 124. In addition, as in the embodiment described above, the parts constituting the prosthesis element can be put into place and locked by moving said parts in a plane that is substantially perpendicular to the common axis of the vertebrae in between which the prosthesis is to be placed.

With reference below to FIGS. 8 and 9, there follows a description of two embodiments of intervertebral implants suitable for use in the spinal arthroplasty system.

In FIG. 8, the implant 140 is constituted, as explained above, by an inter-process spacer 142 and by two straps or ligaments 144 and 146 for securing the ends of the spacer to the spinous processes of the vertebrae. The spacer 142 comprises a body 146 whose ends include grooves or housings 148 and 150 for receiving the spinous processes. The central portion 152 of the spacer body has recesses 154, 156 parallel to the grooves 148 and 150. The purpose of the recesses 154 and 156 is to confer a certain amount of resilience or flexibility to the spacer body and thus allow a certain amount of relative movement between the two vertebrae. Each of the straps 144 and 146 presents a first end 144a, 146a that is secured to the spacer body and a second end 144b, 146b that is engaged in self-blocking locking devices 160 and 162 that can be snap-fastened on the side walls 164 and 166 of the spacer body. This intervertebral implant is described in greater detail in PCT patent application WO 02/071960 in the name of the Applicant.

The intervertebral implant shown in FIG. 9 is described in detail in French patent application 01/15494 in the name of the Applicant.

The spacer 170 shown in FIG. 9 is constituted by two separate parts given respective references 172 and 174. The part 172 defines a top groove 176 for one spinous process, and the part 174 defines a bottom groove 178 for the spinous process of the other vertebra. The inside walls of the parts 172 and 174 define a volume V that is substantially rectangular in shape. Inside this volume V, there is mounted a part 180 that is likewise substantially rectangular in shape and that is made of an elastomer material presenting compressibility properties. The two parts 172 and 174 are held together by a tie 182 engaged in passages such as 184 formed in the parts 172 and 174 in such a manner that the tie 182 completely surrounds the volume V. In addition, each part 172 and 174 of the spacer 170 is fitted with snap-fastening means 184 and 186 for receiving self-blocking systems analogous to the systems 160 and 162 of FIG. 8. These systems enable ties analogous to the ties 144 and 146 of FIG. 8 to be secured tightly so as to secure the spacer 170 to the spinous processes of the vertebrae.

It will be understood that because of the presence of the elastically-deformable block 180, the spacer 170 allows the spinous processes to move towards each other by compressing the part 180. In contrast, the tie 182 limits the possibilities for relative displacements between the parts 172 and 174, thereby limits the possibility of moving the spinous processes apart.

The use of a spacer between the processes presenting a degree of resilience is particularly advantageous, since the at least partial intervertebral disk prosthesis, whether a mechanical ball-and-socket type prosthesis or a disk nucleus prosthesis, allows ball-and-socket type movement between the two vertebrae, which although limited by the spacer, nevertheless remains possible to some extent.

Naturally, other types of intervertebral implant disposed between the spinous processes of the vertebrae on either side of the intervertebral disk for replacement could be used, preferably providing they present a certain limited possibility for relative displacement between the spinous processes of the vertebrae.

More generally, and preferably, in the embodiment of FIG. 8, the recesses 154 and 157 pass right through the middle portion of the spacer, and in the embodiment of FIG. 9, the middle portion of the spacer presents a coefficient of elasticity that is greater than that of its ends.

1-9. (canceled)

10. A spinal arthroplasty system for placing between two contiguous vertebrae, comprising:
an at least partial prosthesis of the intervertebral disk selected from the group comprising ball-and-socket type mechanical prostheses and intervertebral disk nucleus prostheses; and
an intervertebral implant comprising:
a spacer for placing between the spinous processes of the two vertebrae, said spacer having a housing at each of its ends for receiving one of the spinous processes; and
at least one tie for holding each process in a housing of said spacer.

11. A spinal arthroplasty system according to claim 10, wherein said prosthesis comprises:
a ball-and-socket type mechanical prosthesis for the intervertebral disk comprising:
a first assembly presenting a fastener face for fastening to a vertebra and an active face having a portion in the form of a concave first substantially spherical cap; and
a second assembly presenting a fastener face for fastening to a vertebra and an active face including a portion in the form of a convex second substantially spherical cap for co-operating with the first spherical cap.

12. A spinal arthroplasty system according to claim 11, wherein each of said first and second assemblies of said prosthesis includes a fastener element including said fastener face and a prosthesis element including said active face, said elements being provided with mechanical means for securing them together.

13. A spinal arthroplasty system according to claim 11, wherein each prosthesis element comprises two distinct parts for placing side by side, each part having a fastener face and an active face, and in that each prosthesis assembly further comprises means for securing the two parts together, the active faces of the two secured parts defining one of said spherical caps.

14. A spinal arthroplasty system according to claim 10, wherein each prosthesis assembly is constituted by a single part.

15. A spinal arthroplasty system according to claim 10, wherein said partial intervertebral disk prosthesis is constituted by an intervertebral disk nucleus prosthesis.

16. A spinal arthroplasty system according to claim 10, wherein said intervertebral spacer comprises two ends, each including one of said housings, and a middle portion including means deformable in compression that are active when the two vertebrae move towards each other, and tie means for limiting the extent to which the ends of said spacer can move apart under the effect of mutual separation movements between the vertebrae.

17. A spinal arthroplasty system according to claim 10, wherein said intervertebral spacer comprises two ends, each having one of said housings, and a middle portion having a coefficient of elasticity that is greater than that of said ends.

18. A spinal arthroplasty system according to claim 17, wherein said spacer is made out of a single material and its middle portion includes recesses passing right through it.
